(19) United States
(12) Patent Application Publication
(10) Pub. No.: US 2004/0131688 A1
(43) Pub. Date: Jul. 8, 2004

(54) Multi-action particle for structuring biological media

(76) Inventors: Ingman Dov, Haifa (IL); Sarah Dickstein, Ramat Gan (IL); Vladimir Ogenko, Kiev (UA); Alexei Chuiko, Kiev (UA)

Correspondence Address: Edward Langer, Landon & Stark Associates, One Crystal Park Suite, 2011 Crystal Drive, Arlington, VA, 22202, US

(21) Appl. No.: 09/700,496

(22) PCT Filed: May 20, 1999
(86) PCT No.: PCT/IL99/00272

(30) Foreign Application Priority Data
May 21, 1998 (US) 60086261

(51) Int. Cl. A61K 9/127; A61K 9/14

(52) U.S. Cl. 424/489

(57) ABSTRACT

A method for altering the surface structure of ultra-disperse particles to allow predetermined interactions to take place in biological media. In a preferred embodiment of the invention, an ultra-disperse particle is subjected to particle modification, allowing the building of structures on the surface of a basically spherical particle so as to direct its interactions. The inventive method allows the building of protrusions of different shapes and different branching patterns, bonding of different chemicals and changing of electronic structure of the surface on the basically spherical particle. Particle modification enables the formation of layers allowing sequential actions to be performed by the particle, or modification can create more than one type of interactive surface on each particle allowing different interactions to occur simultaneously. These modified particles have applications, for example, as pharmaceuticals, cosmetics, preservatives, and in many other fields. Water-oil emulsions can be created for use in skin creams and other cosmetic and food industry applications. The particles can be used in many application involving radiation to reduce the level of radioactivity necessary, thereby lowering exposure. The particles have the ability to structure biological media by creating a three-sided biological system comprising a biological tissue, the particle and the surrounding liquid.

### FIELD OF THE INVENTION

The present invention relates to chemicals for structuring biological media for use in medical, pharmaceutical, cosmeceutical, agricultural and food industry applications for treatment purposes.

### BACKGROUND OF THE INVENTION

Current awareness of the potential risks involved in the use of many of the health-related products available on the market today has raised the issue of finding more natural solutions to biological problems. Issues such as the overuse of antibiotics, the toxicity of pesticides, and the dangers of radiation treatments have caused the public to become wary of many of the treatments modern research and technology have to offer.

In most laboratories ultra-disperse oxide particles in hydrated form such as fumed silicon dioxide (silica) and other ultra-disperse agents like it are used as common reagents. Ultra-disperse particles are useful for their extremely small particle size (tens of nanometers), a very large surface area and an ability to form chains or networks.

During the process of formation of ultra-disperse oxides the surface of the particles becomes totally hydroxylated (up to a maximum of 7.85 groups per square nanometer) making the surface hydrophilic and capable of hydrogen bonding. Above 110° C. a reversible dehydration of the surface occurs forming, in silicon particles for example, siloxane groups.

In liquid systems, these surface hydroxyls are capable of forming hydrogen bonds forming a network of particles when a sufficient concentration of particles is present. This network increases the viscosity and thixotropy of the liquid. Thixotropy is the time dependent recovery of viscosity after shearing. This allows a liquid with a relatively high viscosity to be sheared and the viscosity temporarily lowered for a specific function and time period. Once the shear force has been removed, the hydrogen bonds will reform the network over time and return the liquid to its original viscosity.

Ultra-disperse particles can be used as suspending agents for suspension of solids in liquids or liquids in liquids (emulsions). The network formed by the hydrogen bonds serves to keep particles separated from each other preventing settling and phase separation.

Although use of ultra-disperse particles in laboratories has become more and more widespread, this use has been limited because the only bonding available on the surface of the particles is the hydroxyl group. A known process exists for practically complete methylation of these hydroxyl groups. Industrial applications have been found for the particles which have been methylated. Their use in biologically-related and pharmaceutical applications is only beginning to be explored.

Thus, it would be desirable to provide a method for changing the surface chemistry of ultra-disperse particles so as to enable different interactions between the particles and the surrounding media, for novel applications in the biological and pharmaceutical fields.

### SUMMARY OF THE INVENTION

Accordingly, it is a principal object of the present invention to overcome the disadvantages associated with the use of conventional ultra-disperse particle preparations and to provide a method for altering the surface structure of such particles to allow predetermined interactions to take place.

In a preferred embodiment of the invention, an ultra-disperse particle is subjected to particle modification. This particle modification allows the building of structures on the surface of a basically spherical particle so as to direct its interactions. The inventive method allows the building of protrusions of different shapes and different branching patterns, bonding of different chemicals and changing of electronic structure of the surface on the basically spherical particle.

Modification can form layers allowing sequential actions to be performed by the particle, or modification can create more than one type of interactive surface on each particle allowing different interactions to occur simultaneously. Particles are constructed such that the result of a first action is anticipated and an appropriate reaction is “programmed” into the particle. Particles can be “programmed” to perform a variety of actions sequentially or simultaneously, producing a multi-action particle.

These modified particles have applications, for example, as pharmaceuticals, cosmetics, preservatives, and many other fields. Water-oil emulsions can be created for use in skin creams and other cosmetic and food industry applications. The particles can be used in many applications involving radiation to reduce the level of radioactivity necessary, thereby lowering exposure.

All types of materials can be used in building the protrusions from the particle surface including, for example, metals, nonmetals, macromolecules, antibiotics, vitamins, microelements, and all types of organic material. These can be removed by chemical reaction with components of the surrounding media or by dissolving them in the media.

The particles can be modified in such a way that the protrusions built on them are highly heterogeneous so that one particle can have the flexibility to deal with many situations. The particles can also be mixed so that some particles are available to deal with a certain type of situation and others are available for different situations. Particle mixtures can be of one material in different sizes or of any mixture of different materials. In this way there exists infinite flexibility in the type of particle which can be created.

The particles have the ability to structure biological media by creating a three sided biological system comprising a biological tissue, the particle and the surrounding liquid. This system stability can be achieved by predetermining the electrical charge of the particles so as to direct them to form an inter-molecular interaction as desired.

A stable three dimensional structure is formed between the system of particles and another component, normally a liquid. The particles bind with the liquid media forming a network which can entrap a third component which may be liquid or solid. With the addition of the third component self-organizing activities selectively act on the nature of the third component building a three component stable structure in which all the parts are functional. The particles can be built in a lock-and-key conformation to make a structure which surrounds the third component. A disturbance of the network is felt throughout the network, much in the way that a spider web transmits motion from the point at which an insect becomes entrapped in the web.

Disturbances in the net can cause localized changes in the viscosity of the media in which the particles are forming the net. For example, the kinetic motion of a live cell will cause a localized change in the viscosity entrapping the cell like a fly in a spider web. This immobilization will biologically inactivate it. The net would not respond to a dead cell or inorganic material.

By way of example, particles can be administered in a powdered form or as a powder pressed into a pill with an anti-aggregation method to allow the pill to be swallowed and then dispersed, for example, by a chemical which causes bubbling. Additionally, particles may be enclosed in a particle in paper bag, such as a tea-bag, to be inserted into water. The tea bag walls prevent dispersion of the particles into the air, so as to prevent inhalation of the particles, but allow free transition of particles through the bag into aqueous media when wet.

Other features and advantages of the invention will become apparent from the following drawings and description.

### BRIEF DESCRIPTION OF THE DRAWINGS

For a better understanding of the invention, with regard to the embodiments thereof, reference is made to the accompanying drawings, in which like numerals designate corresponding elements or sections throughout, and in which:

FIGS. 1a-c show the IR spectrum of particles during the methylation process at 0, 10 and 30 min, respectively;

FIG. 2 is a photograph of the network formed by modified ultra-disperse particles in an aqueous solution;

FIG. 3 is a graph of the number of boxes of size 1/n needed to cover the fractal;

FIG. 4 is a photograph of a network of modified ultra-disperse particles and a finer network of TiO₂ particles;

FIGS. 5a-c show partially methylated particles, 25% 50% and 75% methylated, respectively, modified with the addition of TiO₂, Al₂O₃ and SiO₂;

FIG. 6 shows a table of types of particle modification possible along with mechanisms and possible applications;

FIG. 7 is a photograph of a bacterium surrounded by ultra-disperse particles;

FIG. 8 is a table of results from microbiological experiments involving particle effect on bacterial growth;

FIG. 9 is a histogram of bacterial colony area as affected by application of ultra-disperse particles;

FIGS. 10a-b show respectively, tables of data from toxicity studies testing the levels of chloride and β-lipoprotein in the blood of rats treated with ultra-disperse particles;

FIG. 11 shows a table of the alteration of sensitivity to antibiotics when administered in conjunction with an ultra-disperse particle treatment;

FIG. 12 shows a table of the results of treatment of patients with purulent inflammatory diseases treated with an ultra-disperse particle treatment;

FIG. 13 shows a table of regression of clinical manifestations and normalization of laboratory indices on the fifth day of treatment with ultra-disperse particles;

FIG. 14 shows a table of the impact of ultra-disperse particle treatment on wound microflora sensitivity to antibiotics,

FIG. 15 shows a table of clinical laboratory index dynamics for patients with periodontitis; and

FIG. 16 shows a table of mineral modifications and their medical applications.

### DETAILED DESCRIPTION OF A PREFERRED EMBODIMENT

Ultra-disperse particles of hydrated oxides have different electrical potentials allowing them to interact with other surfaces. It would be desirable to modify the surface of the particle to provide a template for different chemical and physical interactions. The prior art has demonstrated the ability to modify the surfaces of ultra-disperse particles but this has been limited to a process of almost complete methylation (for example, De Gussa Corp., Aerosil R812 and Aerosil R972).

The present invention provides a means of modifying the surface of ultra-disperse particles of hydrated oxides based on a method for partial methylation of the particle surface, followed by further modifications as desired.

In the first stage, the particle is methylated for up to 60 minutes, depending on the desired percentage of methylation. The surface hydroxyl groups which appear approximately every 7 angstroms on the surface of the particle, are partially replaced by methyl groups in a well known process, by exposing SiO₂ to methyl-chloride-silane or cycled organic poly-siloxane D4-D8 in the gaseous phase or other functional organic molecules such as spirits, glycols, phenols, etc. The percentage of the surface which is methylated and becomes hydrophobic depends on the time of exposure, concentration of the active molecules and reaction temperature. The production process is as follows:

1) the “base” (ultra-disperse particles suspended in an aqueous medium) is heat treated in an open vessel (in air) at 200°, 400° or 650° C. for SiO₂ and at 200-400o° C. for Al₂O₃ and TiO₂. This removes the physically absorbed water and bound structural water.

2) After the heat treatment, the substance is reacted with the appropriate reagent in the gaseous phase (dimethyltrichlorosilane, trimethyltricholosilane, polysiloxanes, cyclosiloxanes, oligomers, etc.) This reaction is allowed to occur for between 5 min to 1 h depending on the desired substitution level, at 200-300° C.

3) The excess reagent and reaction products are removed. This is followed by hydrolysis of the unreacted chloride groups on the surface, effected through heating at 250-300° C. for 1 h in the presence of saturated water vapor.

4) After removal of the reagent and reaction products, heating is carried out in an open vessel (in air) or in an inert atmosphere (with nitrogen blown through the reactor) at 200-300° C. It is followed by cooling at room temperature and discharge.

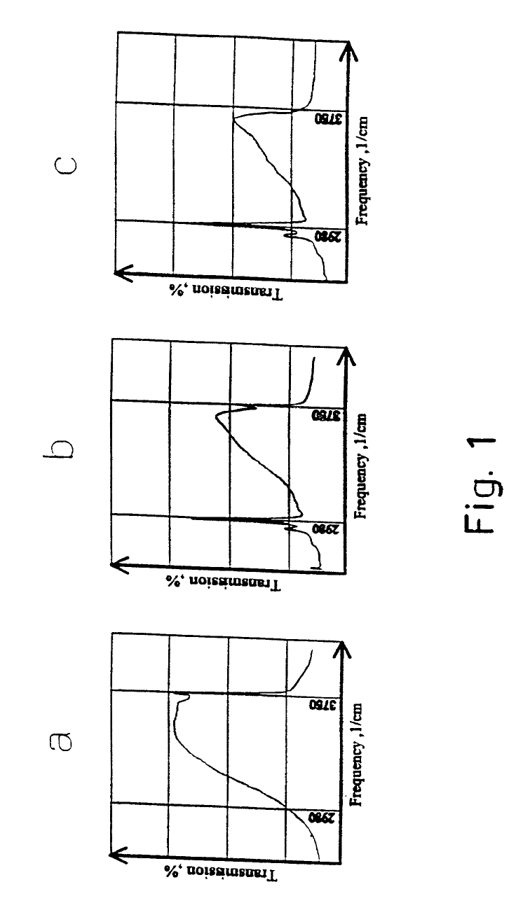

As seen in FIGS. 1a-c, percent methylation can be ascertained by checking the IR spectrum, with the peak for hydroxylation appearing at 3750 nm and the peak for methylation appearing at 2980 nm. The reaction can be quantitatively controlled by IR spectroscopy since the intensity of characteristic lines of absorption of covalent bonds corresponds to the substitution of the structural OH groups on the surface by Si-methyl radical groups. Typical temperatures for the reaction are in the range of 100-300° C. FIG. 1a shows the IR spectrum at 0 min of exposure. No peak is seen at 2980 nm because no methylation has occurred. In FIG. 1b, the IR spectrum for an exposure of 10 min. at 250-300° C. provides approximately 50% surface hydrophobicity without any organic catalysts in the gas, as seen by the sharp peak at 2980 nm. This partial methylation provides a particle which is partially hydrophobic and partially hydrophilic. In FIG. 1c a 30 min exposure has provided greater methylation.

In this way the particle can be provided with hydrophobic and hydrophilic modified surfaces to form non-organic amphiphilic systems which can interact with membranes in a manner similar to peptides. This structure can form discrete ion channels and affect the cellular potential to change its ion or chemical permeability, or even destroy the biological membrane, causing cytolysis. The part of the surface which will be hydrophobic or hydrophilic can be provided ranging from 10-90% as per the application.

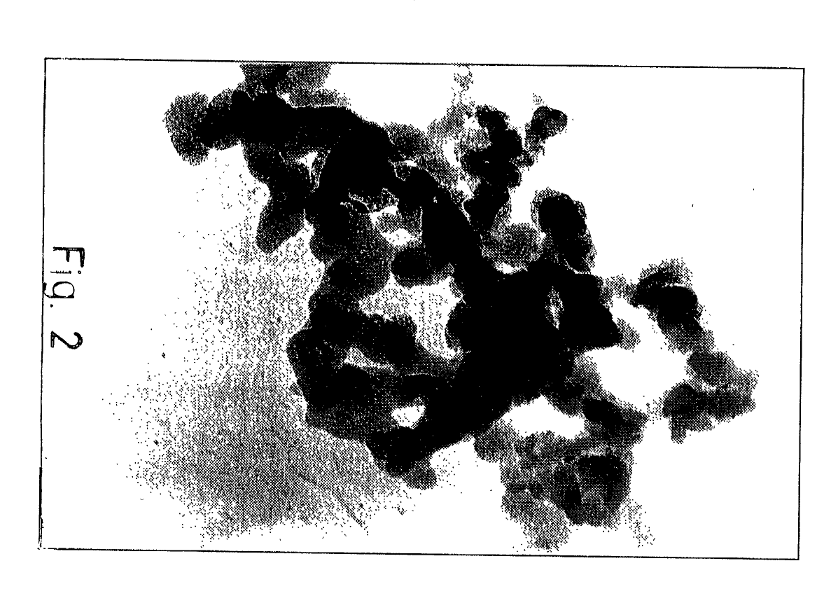

Referring now to FIG. 2, there is shown a network of modified ultra-disperse particles formed in an aqueous solution. This ability of even unmodified ultra-disperse particles to form a network allows rheology control, increases viscosity and produces thixotropic behavior. The hydroxyl groups on the surface of the particle attract water.

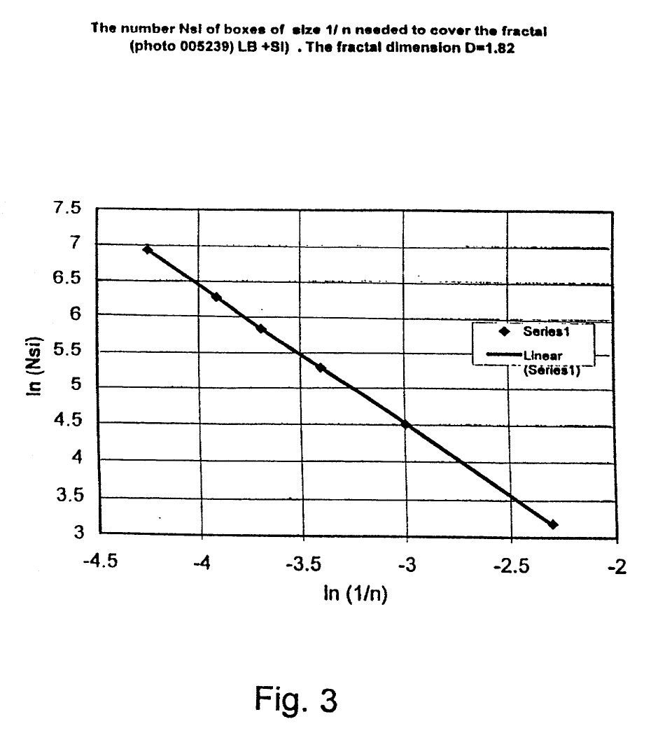

As seen in FIG. 3, the particles have a high fractal dimension producing highly stable structures. As box size is decreased, length increases indicating that the particles form a fractal structure, with a fractal dimension (D) of 1.82 as shown in the graph in FIG. 3. This enables the particles to self-adapt to the element they are “programmed” to pick up.

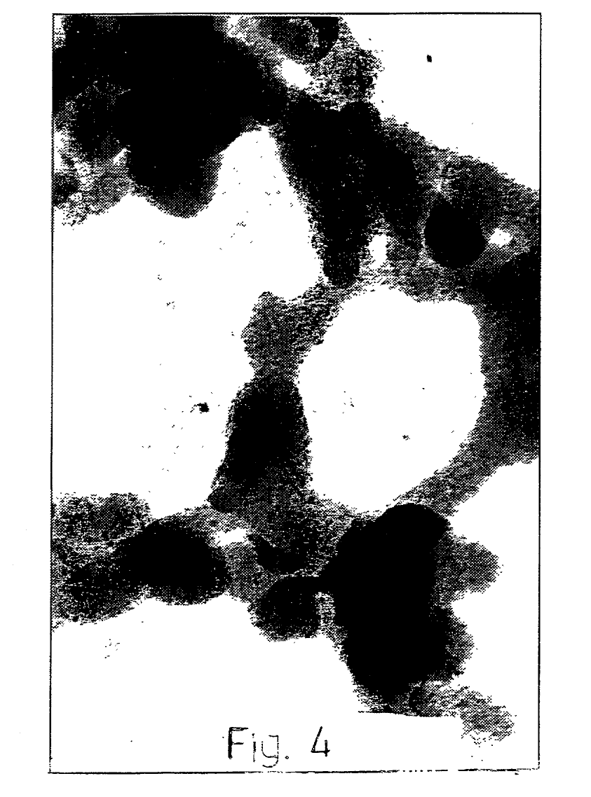

In FIG. 4 there is shown a network of modified ultra-disperse particles enclosing particles of TiO₂. In the process of modifying the oxide particles so that they will have titanium modifications on them, free active titanium particles are produced which are smaller than any currently producable. These smaller particles form an even finer network of their own, seen in the spaces between the larger, darker net. The patterns which are created are more dense than any existing semiconductor device and are of an order smaller than any other existing particles.

With progressive methylations, the attraction of the water is reduced, until the field of the hydrophobicity surrounding the particle will no longer tolerate water in the surroundings. Since water cannot attach to the hydroxyl groups, these active OH groups are left open to make their strong bond with whatever other chemical is provided. Using this hydrophobic field the surrounding water is structured to make a net of different fractal structures.

A hydrophilic-hydrophobic combined particle can bind liquids of opposite nature, for example, oil and water, and provide a stable thixotropic water-oil emulsion. A partially hydrophobic, partially hydrophilic particle can act as a linking agent to link together hydrophobic cells with hydrophilic cells to form an emulsion. The template with the hydrophobic and hydrophilic ratio (K) can control the structural and Theological properties of both the system and the emulsion as a whole. This technology allows creation of almost “non-creatable” materials, such as an emulsion of oil and water without alcoholic components, which are the traditional emulsifiers. In addition, the features of each component are modulated by the features of the particle, such that new effects are created because of the combination. Maximal homogeneity of the emulsion is achieved for K corresponding to the proportion of the hydrophobic component (e.g. oil) and water. The content of the particles has an upper limit which can be estimated by the need for blockage of all the hydrophobic surfaces by oil, otherwise water can not be inserted into the system.

A water-oil emulsion is provided by encapsulating water droplets in a layer of ultra disperse hydrophobic particles with or without hydrophilic particles (less than 5%, possibly on the order of 0.1%). These particles are passed through an ultrasound atomizer, with a usual drop-size of 50-100 microns. These drops are fed into a chamber onto a layer of hydrophobic particles and are coated by them with the aid of collision forces. The coated particles are then introduced into the emulsion under turbulent mixing. The hydrophilic particles will structure the water and the hydrophobic particles will allow insertion into an oily medium so that the resulting emulsion will contain an extremely high water content.

This emulsion has many uses, including for example, the production of programmable particles for use in skin moisturizers in the cosmetics field. If an emulsion of water in an oily base is provided, when the cream is massaged into the skin the droplets of water coated with hydrophobic material will break open within the case of oil which will be attracted to the oily skin, supplying either oil or water as needed. If the skin is dry, the oil will be attracted to the skin. If the skin needs water the droplets will be attracted to the skin. Thus, the skin is provided with the treatment that it needs.

The hydrophilicity or hydrophobicity of the particle can be used as a response to bacteria. For example, the use of a hydrophilic particle will attract water and structure it so that there is no free water available to the bacterium. This in essence freezes the bacterium within a block of structured water, disrupting any communication between the bacterium and the surrounding medium.

This bactericidal effect makes the particles useful as safe and effective preservatives and stabilizers. A wide variety of particles can be used for a broad spectrum protection, in a much lower concentration than conventional preservatives and stabilizers. This use is especially important in cosmetics, where the level of cleanliness needed for medications is not observed, and creams are used repeatedly by insertion of non-sterile fingers into the containers. Silica is currently being used in this industry in high percentages. Use of the modified particles would significantly reduce the amount needed to function as a preservative below levels known in the market today.

Once the degree of methylation has been attained, further modification can be accomplished. Because methyl groups are difficult to modify, the methyl groups act as caps to the sites which have been methylated, allowing further modification of the hydroxylated sites without modification of the methylated sites, if desired.

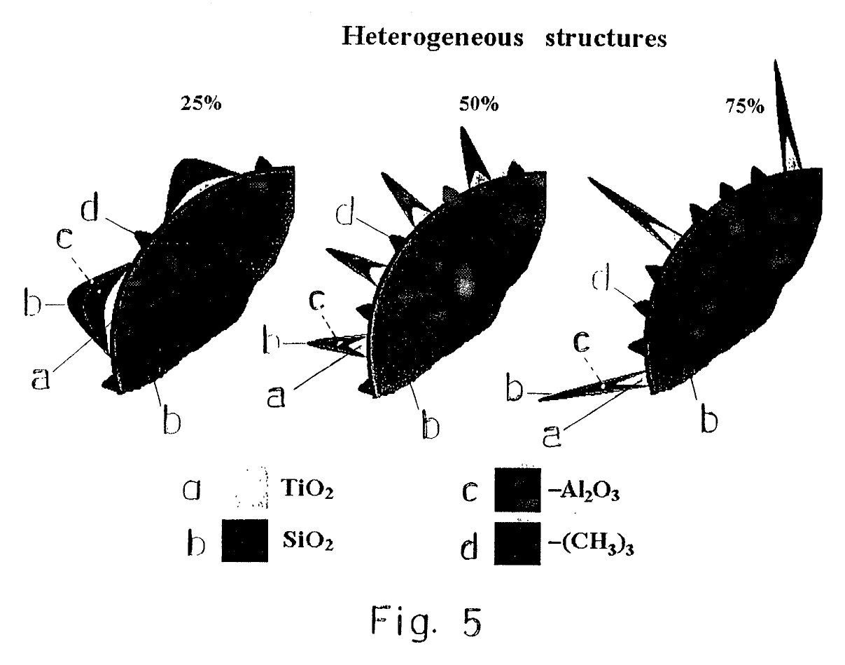

As can be seen in FIGS. 5a-c, these sites can be selectively built on so as to control the structure and the chemical reactivity of the particle. Additions can be selected to modify surface charge, pH and electrical potential. Protrusions from the surface can take the shape of wide or narrow spikes or can branch. In FIG. 5a, 25% methylation has occurred leaving 75% of the surface available for modification. Wide protrusions have been formed with the addition of TiO₂, Al₂O₃ and SiO₂ in successive layers to the modification sites. In FIG. 5b 50% methylation has occurred leaving 50% of the surface available for modification. In FIG. 5c 75% of the surface has methyl caps on it leaving room for narrow spiky protrusions formed by the addition of the same metals, TiO₂, Al₂O₃ and SiO₂, on the other 25%. The protrusions can be built to size specifications so as to capture a virus-sized particle or act as a chelating agent. The more the surface is methylated, the less opportunity is available for modification. As more surface is methylated the protrusions will be of smaller sizes and therefore more needle-like. These narrow-based protrusions will be long and high and the density of the protrusions per area will be lower.

These protrusions can be non-uniform on the surface of the particle with different protrusions being built and capped at different times for maximum flexibility of the system so as to react selectively in different environments. In order to build a second type of protrusion the particles are heated to between 500-700° C. to demethylate the capped sites on the surface of the particle. Because of the high electrical gradient of the spike protrusion the spike protrusions will become methylated, in effect capping the spikes and leaving open hydroxylated sites on the surface of the particle. These sites are now built on with another sequence of materials and shape formations. Particle modification can take place in many steps creating a particle which has a sequential release of different layers of coatings A dissolvable structure can provide a slow-release mechanism. These highly heterogeneous particles have the ability to deal with different states in a selective manner.

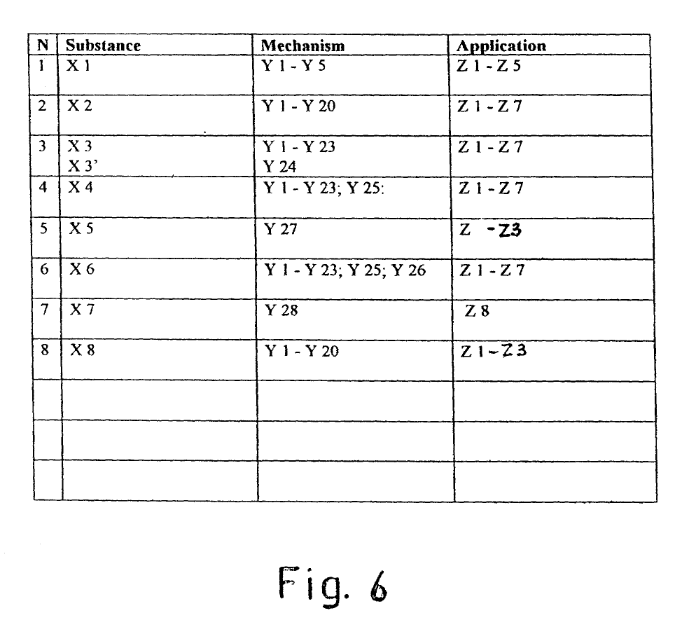

In this way, an infinite combination of particles and modifications can be developed for any specific cause. FIG. 6 shows a table of some of the different types of particle modifications possible, along with mechanisms of action and possible applications. In column 1 substances are particles modified as follows:

X1 are ultra-disperse oxides such as SiO₂, Al₂O₃ and others in hydrated form.

X2 are ultra-disperse oxides with a given hydrophobic-hydrophilic balance on the surface.

X3 are ultra-disperse oxides with non-uniform heterogeneous structures.

X3′ are ultra-disperse oxides with needle structures capable of separation of phases. They are hydrolytically unstable so that the protrusions are able to detach in aqueous solution providing an additional net of much smaller particle sizes (see FIG. 4).

X4 are ultra-disperse oxides with “island-mosaic” inclusion and formation. These particles are covered with islands of different modifications which can bind different components.

X5 are mechanical mixtures of ultra-disperse oxides in given correlations.

X6 are ultra-disperse oxides with functional groups capable of chelation.

X7 are ultra-disperse oxides with stalactite or spiked structures.

X8 are ultra-disperse oxides which act as carriers of additives such as antibiotics, vitamins, microelements, poisons and other compounds.

In column 2 of the table in FIG. 6, the mechanism of action of the particles as shown in column 1 is explained.

In this column the following key is used:

Y1—ultra-disperse oxides acquire a charge through a double electric layer and are also capable of electrostatic interaction with regions of a third component.

Y2—these particles are smaller than the bio-objects and are capable of electrothermophoresis and other specialized interactions.

Y3—ultra-disperse oxides can undergo charge reversal depending on the pH of the environment. For example, Al₂O₃ acquires a positive charge at pH 2-8 and a negative one above pH 9.

Y4—the electrostatic interaction of ultra-disperse particles of different natures can be used for directed action on microorganisms of different types.

Y5—ultra-disperse particles are capable of interaction with affected cell regions or with bacteria, while retaining their high absorption capacity and their selectivity.

Y6—the evolved active surface of the particles takes up the toxic substances formed as a result of the vital activity and decomposition of the biosystem. Their elimination can be effected selectively by modifying the surface chemistry.

Y7—ultra-disperse particles are always of dual action, i.e. any biological function caused by their presence or by interaction with them is followed by a process of possible toxic result absorption, neutralization or removal, i.e. action and deactivation of the system's toxic response.

Y8—ultra-disperse particles of a given surface chemistry and structure are characterized by a broad interaction spectrum, from intermolecular to chemical, either with the environment or with the boundary of any system located in it. These interactions result in the formation of a three bond network imparting stability to the network through the broad spectrum and the charge states of the particles.

Y9—on appearance of a third component in the system, the equilibrated structures formed earlier exhibit active, self-organizing properties, thereby responding adequately and selectively to the appearance of this third component and to its charge state, thereby forming a localized stable three component system. This system is capable of realizing the desired final result through linking of the different active centers (islands of different types of modifications) and the components on the particles, so that the particles function as linking points between the components in the formation of the network.

Y10—the selectivity of particle action depends on the size and shape of the object, on the charge, on the hydrophilic-hydrophobic pattern and on the availability of functional groups. Ultra-disperse particles can act on a broad or narrow front, are capable of separating living matter from inanimate matter, different types of living matter, and solid from non-solid and can recognize on object and ignore another.

Y11—ultra-disperse particles permit structurization of the bioenvironment with formation of locally non-homogeneous regions or nano-size fluctuations, interacting through the network of three dimensional bonds containing the inorganic particle.

Y12—the structured thixotropic biofluids are analogs of membranes impeding the transport of bacteria, of their nutrients and of dissolved inorganic compounds and ions.

Y13—in the thixotropic environment, the particles are capable of reacting variously with a living or an inanimate third component. In the case of the inanimate component a stable three dimensional structure is formed. In the case of a living third component an unstable structure is formed which has variable thixotropy modulated by the mobile living component. The latter can be differentiated through the degree of modulation.

Y14—the capacity of ultra-disperse particles to be adsorptive and chemisorptive and their ability to form chelates allow inorganic and organic components to be isolated.

Y15—the ultra-disperse particles acquire adsorptive capacity for interaction with hydrophobic-hydrophilic regions of the bio-objects as well as for specific interaction with components of the living environment such as adsorption of proteins, structuring of water and mobilization of organic and inorganic compounds.

Y16—a combination of positively and negatively charged particles can lead to encapsulation of bacteria. Creation of a given hydrophobic-hydrophilic level can increase this effect.

Y17—with the aid of hydrophilic particles bacteria can be inactivated (“frozen”) inside a block of structurized water, with practical disruption of the link between the bacteria and the environment.

Y18—hydrophobic particles can be used for intermolecular interaction with hydrophobic regions of membranes, as well as for supply and removal of oils.

Y19—creation of a specific hydrophobic-hydrophilic balance on the surface of the ultra-disperse particles permits formation of a branched three-dimensional network in a system of non-interactive hydrophobic-hydrophilic environments across the surface of a solid body. The structure can form discrete ion channels and affect the cellular potential to change ion or chemical permeability or even destroy the biological membrane causing cytolysis. The part of the surface which will be hydrophobic or hydrophilic (the K ratio) can be provided ranging from 10-90% as per the application.

Y20—a hydrophobic-hydrophilic particle can bind liquids of opposite nature, for example, oil and water, and provide a stable thixotropic water-oil emulsion. The template with ratio “K” can control the structure and rheological properties of both the particles and the emulsion as a whole. This technology allows creation of almost “non-creatable” materials, such as an emulsion of oil and water without the traditional emulsifiers.

Y21—using a surface with a given hydrophobic-hydrophilic balance and causing chemical reactions over specific surface hydroxyl groups with metal chlorides such as AlCl₃, TiCl₄, etc., highly non-uniform heterogeneous environments are created with new thixotropic properties, different charges, different photochemical abilities and other changed properties. Opposite charges are obtainable on the same particle.

Y22—a reaction with a given cycle (e.g. chemical inoculation—chloride hydrolysis) yields nano-size formation of various oxides on the surface of the ultra-disperse particles, as well as combination of the oxides such as: SiO₂—SiO₂, SiO₂—TiO₂, Al₂O₃—SiO₂, TiO₂—SiO₂ and others.

Y23—the programmable particles can be formed with a series of layers of active ingredients which are encapsulated in slow-release covers. The multi-level action can be programmed with active ingredients being released in sequence and the final active ingredient being programmed to absorb the results of the reaction.

Y24—after the stratification of the chlorides (see methods) and interaction with the aqueous environment over the bonds SiO₂—Ti(O)₃, the ultra-small particles on the surface are capable of separation and electrostatic interaction forming their own smaller network (see FIG. 4).

Y25—the spatial structures posses a suitable “lock and key” system whereby the ionic channel is shut, thus encapsulating the microbe and shielding it from the environment.

Y26—replacement of the structural hydroxyl groups with other groups such as inorganic and/or organic radicals (amines, alcohols, iodine, bromine and other bioactives) leads to formation of bonds of the donor acceptor type, complexes with coordination type charge transfer, covalent bonds and dispersion interaction with the functional radicals of the bio-object.

Y27—oxides in mechanical mixtures are differently charged in the presence of water, depending on the pH of the environment, and therefore will interact differently with each other and with specific biomembrane regions.

Y28—mechanical mixing, followed by settling of substances with heterogeneous structures in an aqueous environment leads to formation of xerogels with an ultra-heterogeneous pore structure. These gels posses an intrapore structure with a vastly developed labyrinth.

Column 3 in the table in FIG. 6 shows possible applications of the particles in column 1, corresponding to the following listing:

Z1—Medicine

Z2—Cosmetics

Z3—Hygiene

Z4—Food industry

Z5—Agriculture

Z6—Purification of water

Z7—Sterilization of water

Z8—Disinfection

Following are examples of some of the methods of production of the various types of particles. A description of the production of X2 particles has already been given in the opening of the description.

X3—Building on the X2 structures, reactions are effected over residual unreacted hydroxyl groups with chlorides of the desired metals (AlCl₃, TiCl₄, etc.). For example, pyrogenic silicon oxide with 30% structural hydrophilic groups is heated to 200-250° C. for 1 h. A reagent (one of the chlorides) is added, 10% by weight. The reacting mass is held in chloride vapor for 1 at 200-250° C. This is repeated up to 5 times.

X3′—Building on the X3 particles, after application of the chlorides and interaction with the aqueous medium, the particles are capable of separation and electrostatic interaction,

X4—Building on X2 particles with 10-30% hydrophobic groups, the remaining 70% are substituted for Al₂O₃, TiO₂ at 200-400° C.; for SiO₂ at 200°, 400° and 650° C. The reaction is controlled through the IR spectrum. A possible alternative base is an X3 substance (with metal chlorides). The samples are then heated from 400-700° C. (thermal destruction of hydrophobic groups) and interacted with any oxides in water vapor (the vapor blown through) or in air.

X5—Initial base —SiO₂ (10-90%) and Al₂O₃, TiO₂, Fe₂0₃ etc. mixed in air at room temperature. The same ingredients can be heated to 200-400° C.

X6—Building on a base of X1 to X4 substances, structural hydroxyl groups are replaced by other inorganic and/or organic radicals (amines, carboxyls, alcohols, iodine, bromine), antibiotics, vitamins and other bioactive compounds. For example, instead of the water-vapor hydrolysis stage, ammonia is blown through at temperatures from room temperature to 200° C. for 1-2 hours, yielding Si—NR or Si NHR groups where R is H, CH₃ C₂H₅, C₃H₇, C₄H₉. Phenol (as antioxidant) can be used instead of ammonia.

X7—substances with heterogeneous structures, mixed mechanically in an aqueous medium at room temperature.

X8—ultra-disperse particles as carriers for small amounts of bioactive additives such as drugs, trace elements, vitamins, poisons, etc.

As can be seen from FIG. 6, the possibilities for modification and application of the modified and unmodified particles are endless. Following are some illustrative examples.

It is known that a wound in the body will cause a localized change in the electrical potential from a negative to a positive charge. In general, the bacteria which cause infection in the body have a negative electrical potential. The bacteria, therefore, are electrically attracted to the wound site, thus providing them with an entry to the body to insert their toxins. It would be desirable to provide a method of blocking this entry so as to prevent toxins from entering the body.

For example, using the fact that the electrical potential at a wound site is changed from negative to positive, if one wishes to protect the body from bacterial toxins at this entry point, a negatively charged particle (of the X1 type) is used to coat the wound site and change the potential. The particles used are much smaller than the size of a bacterium, and therefore are able to fit between the bacteria and reach the wound site. The extremely small size of the particles creates a very large percentage of active surface. Once the wound site has been coated it is no longer a site for insertion of toxins, nor does it attract the negatively charged bacteria. For this purpose, surface nano-particles of SiO₂ or TiO₂ can be used as they have a negative charge in water.

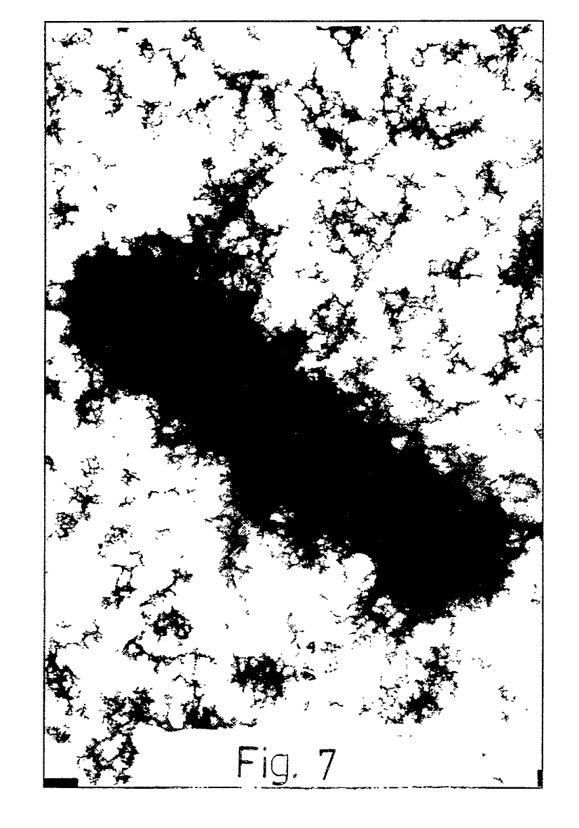

Alternatively, a particle which is positively charged in water, such as Al₂O₃, is attracted to the negatively charged bacteria, as seen in FIG. 7. This photograph shows the interaction between the particle and the bacteria, effectively coating the bacteria, thereby neutralizing it. It can neither release toxins nor can it pick up material from the surrounding media. The particle-bacterium combination then remains within the biological system inertly until it is flushed out. A combination of positively and negatively charged particles can be used to both coat the wound site and encapsulate the bacteria for a complete effect.

The system is self-regulating, because the negatively charged particles will remain attracted to the positively charged wound site until the wound heals and the potential of the site returns to its normal negative charge. Once the wound is healed the negatively charged particle will no longer be attracted to the site and will be flushed away. This occurs as a natural progression with the healing of the wound. In addition, the treatment can be used without positive diagnosis because if there is no need for treatment there is no effect of the particle.

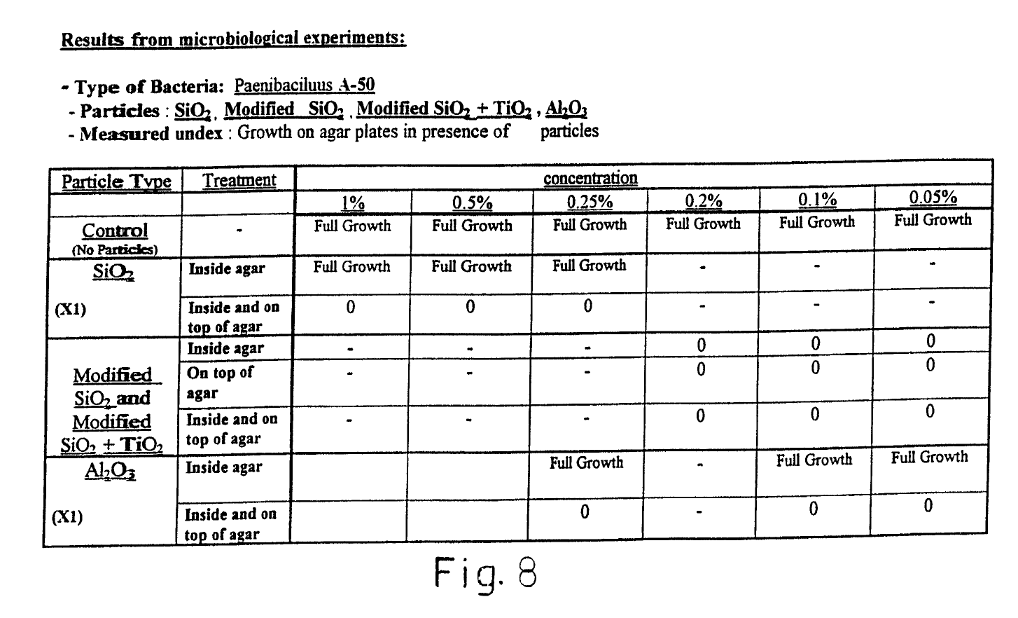

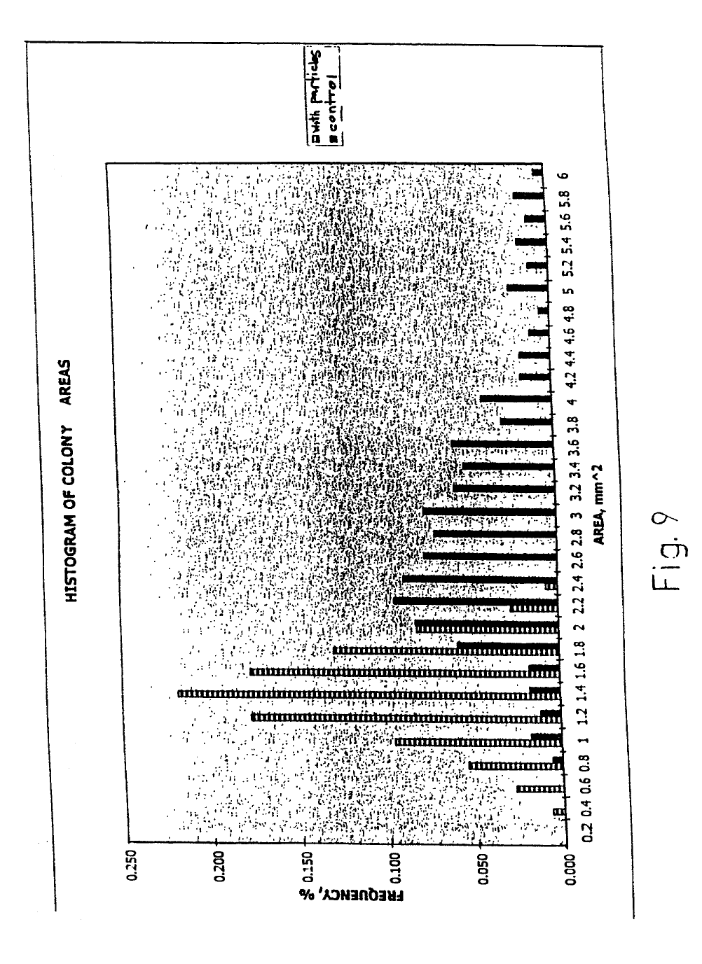

This bactericidal effect has been shown in FIG. 8, which shows a chart of the results of microbiological experiments performed on Paenibacillus bacteria. In the first row, a control set of examples is shown in which full growth was achieved on the surface of all petri dishes. In the second row, plates were poured and unmodified SiO₂ (X1 type particles) was added to the agar. This did not have an effect on the growth of the bacteria. However, when the particles were added to the agar and smeared across the top of the agar in various concentrations (1%, 0.5%, 0.25%), in all cases growth of the bacteria was completely stopped and 0 growth was recorded (as seen further on in FIG. 9). In the third row, when modified SiO₂ particles or modified SiO₂—TiO₂ particles were used either in the agar alone, on top of the agar alone or in a combination of the two methods full arrest of growth was recorded, even at the lower concentrations of 0.2%, 0.1% and 0.05%. This shows a much higher efficacy of the modified particles over the unmodified particles. In the fourth row unmodified particles of Al₂O₃ were tested, showing similar results to the other X1 type particles shown in the second row. The particles added only to the agar were ineffective but when used in combination with smearing on top of the agar full arrest of growth was attained even at the lower concentrations of 0.25%, 0.1% and 0.05%.

In FIG. 9 we can see the arrest of bacterial colony growth with the application of particles. The solid bars represent the normal curve shown by bacterial colonies, a double phased curve. The hatched bars represent bacterial colonies treated with the particle which show a single, narrow bell curve in which none of the colonies reached an area above 2.4 mm², as opposed to the untreated colonies which were as large as 6 mm².

By building different structures on the surface of the particle, a particle can be programmed to respond to certain biological elements. It can be directed at a certain part of a specific type of bacteria. For example, particles can be directed to attach themselves to the flagella of a bacteria, thereby immobilizing a bacterium without lysing it.

The spike protrusions formed on the surface of the particle are of an appropriate size to be inserted into the ion channels of cell membranes. They can be constructed with a material on the tip for insertion into a cell. Upon insertion of the spike through the ion channel the material is released into the cell. In this way, the spike functions like a needle to inject material into a living cell.

Another preferred embodiment involves forming particles with a spatial representation that gives a lock and key fit to block ion channels of a given diameter in the cell membrane (mechanism Y25). This in effect encapsulates the microbe preventing its communication with the medium.

These particles are useful both in an ingested form and in a powder for sprinkling on open wounds such as burns. In the ingested form, the powder can be pressed into a pill and provided with a dispersing factor to allow the pill to be swallowed and then dispersed, for example, by a chemical which causes bubbling. In an open wound, the powder prevents infection, allowing exposure of the skin to the air thereby allowing the skin to heal more quickly.

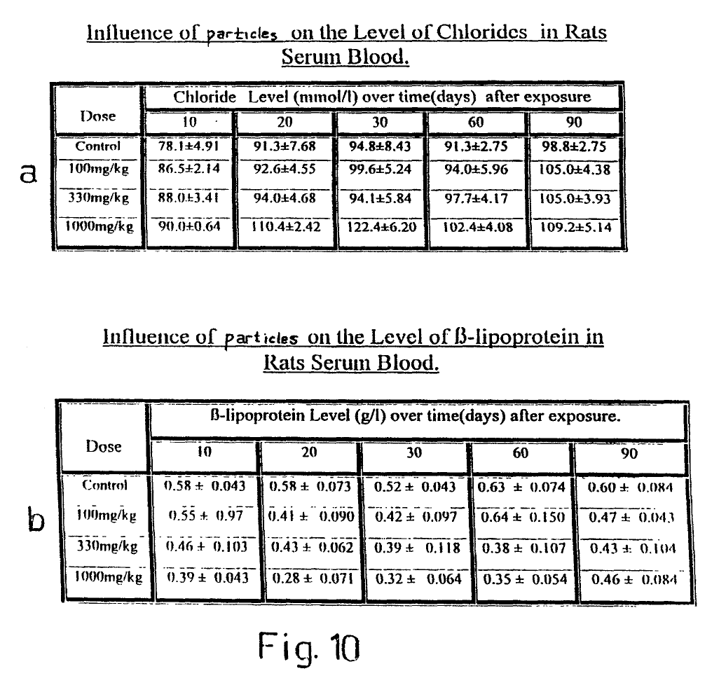

As seen in FIGS. 10a-b, standard toxicity studies have shown the particles to be safe for use as a drug treatment. Shown in FIG. 10a is a table with the results of tests for chloride levels in the blood at 10, 20, 30, 60 and 90 days of exposure, at three different dosages of the particles in rats. Chloride levels remained acceptable throughout. In FIG. 10b the table shows the levels of β-lipoprotein which were tested at 10, 20, 30, 60 and 90 days of exposure at the same three dosages of the particles in rats as in FIG. 10a. β-lipoprotein levels remained acceptable throughout. Not shown are results of other standard toxicity studies which were all deemed acceptable, including levels of vitamin C, inorganic phosphorous, alkaline phosphates, urea, and creatinine.

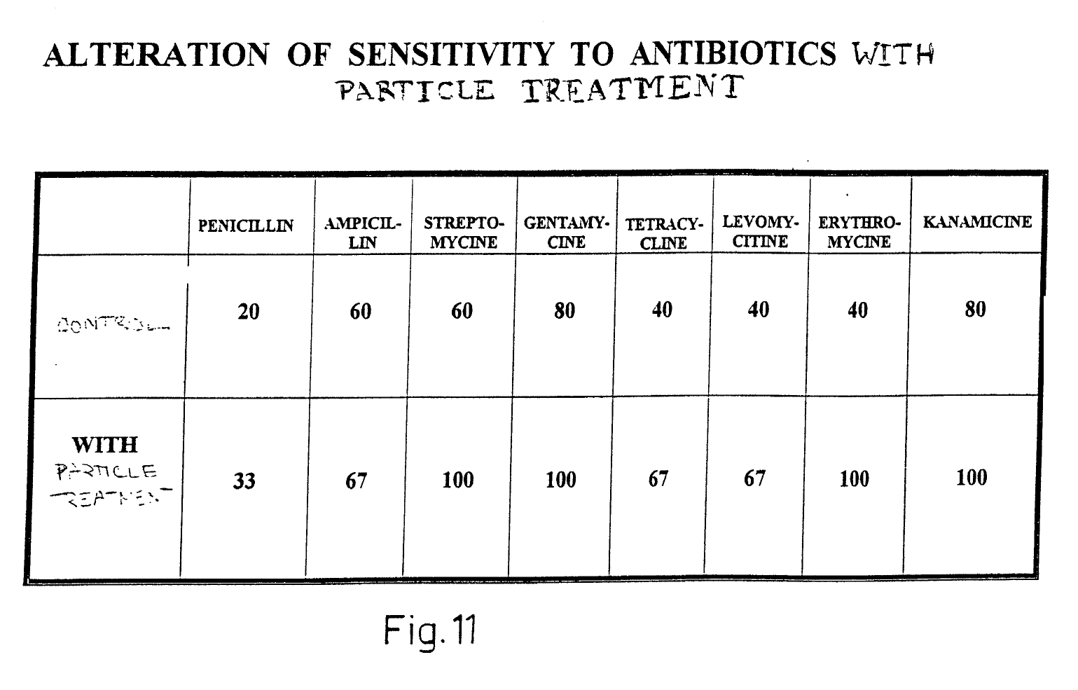

FIG. 11 shows the alteration of patient sensitivity to antibiotics under treatment with an ultra-disperse particle. In the first row, there are shown the sensitivities to treatment in a control group, treated only with the particles. In the second row a second group of patients was given treatment with the same antibiotic with the addition of treatment with ultra-disperse particles. It is clear that in all cases sensitivity to antibiotics is boosted with the use of the ultra-disperse particle. This enables a more effective use of antibiotics and will allow the patient to use lower doses. A body will release toxins in response to a major stress such as an infection or a heart attack. The particles bind the toxins released by the infection and by the body in response to the infection, giving a general cleansing effect. Therefore, there is less need to activate the immune system giving the body more strength to heal itself in a shorter time.

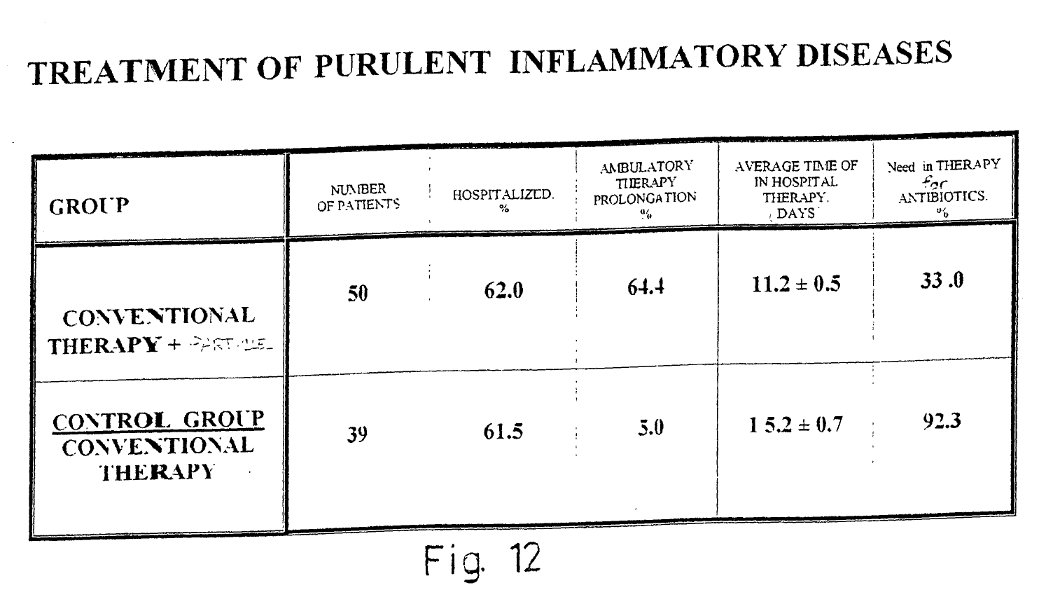

Similarly, FIG. 12 shows the results of treatment of patients with purulent inflammatory diseases with conventional therapy and with conventional therapy and the ultra-disperse particle treatment. As shown in the first row, patients who received the ultra-disperse particle treatment in addition to the conventional treatment spent less time in the hospital and significantly fewer required antibiotic treatment than those who received only conventional therapy (second row). In addition, post hospitalization ambulatory therapy was of a shorter duration.

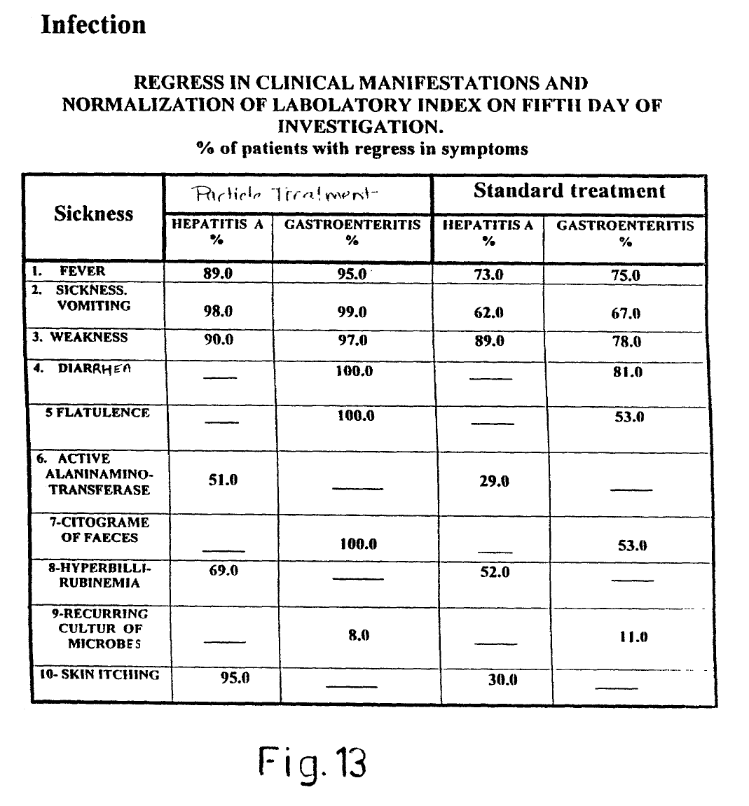

FIG. 13 shows the results of a study done on regression of clinical manifestations and laboratory indices after five days of treatment. Patient groups included those suffering from hepatitis A or gastroenteritis. In a series of ten symptoms listed in column one those treated with the ultra-disperse particle treatment all showed a higher percent of regression in these symptoms than those in the control group which only received standard treatment.

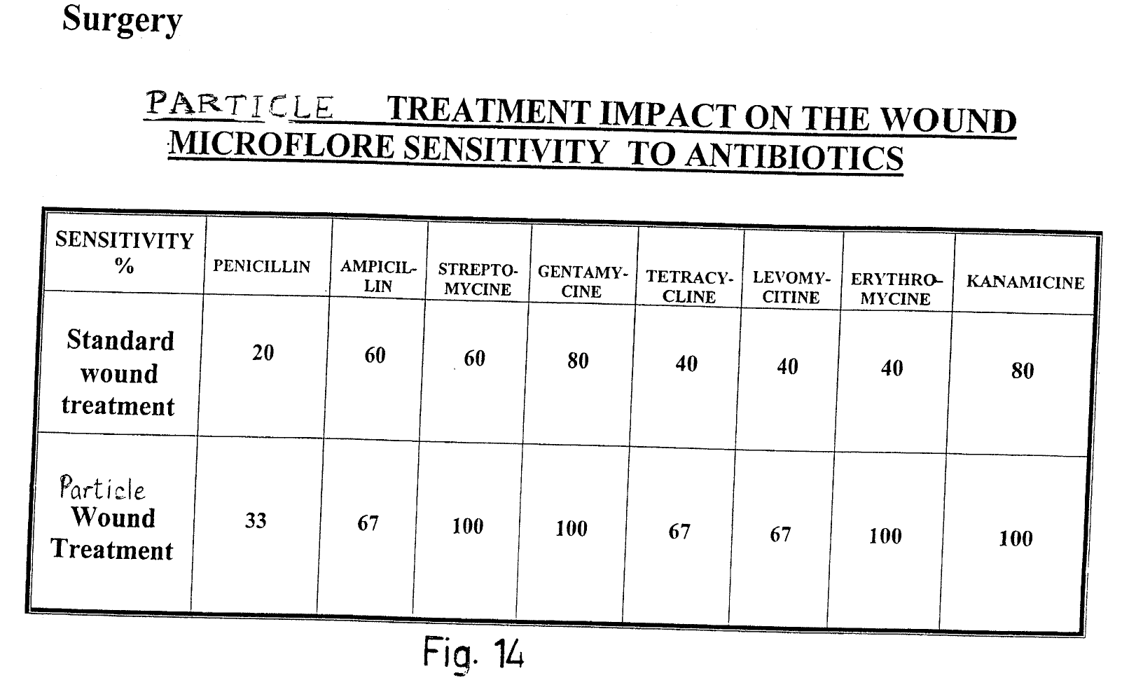

In a study of wound treatment, shown in FIG. 14, eight antibiotic treatments were used on a control group to show sensitivity to the standard treatment, as shown in row 1. In row 2, the patients received particles modified to carry the named antibiotics. In every case efficacy of the antibiotic was boosted in response to the use of the ultra-disperse particles.

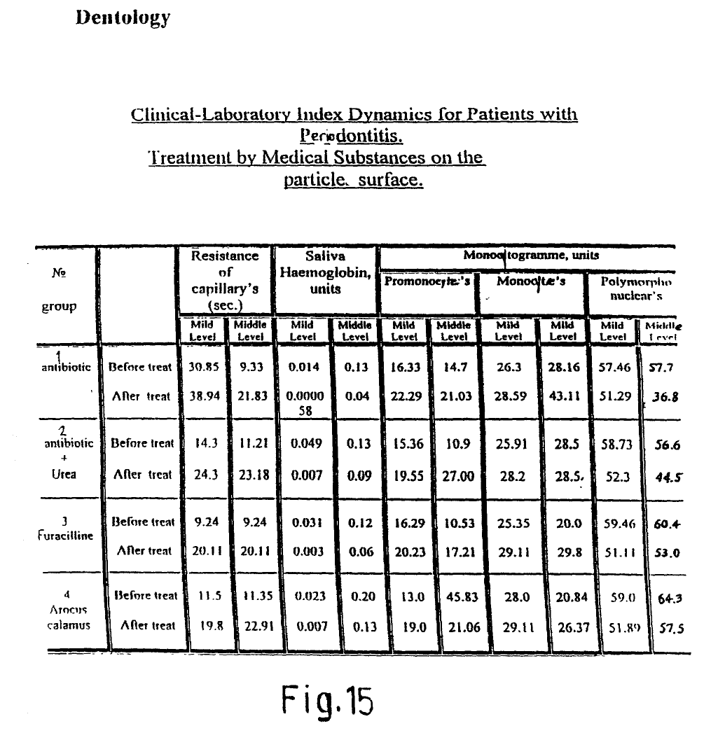

In dentology studies, particles were modified to carry antibiotics that are used in the course of standard periodentistry treatments, as shown in the table in FIG. 15. Four different standard procedures were used, as shown in column 1 because gums are not always sensitive to the same treatments. Two groups of patients were used, those with a mild severity of gum disease and those with a moderate severity level of disease. Three tests were done on each patient: (1) resistance of capillaries in seconds, which is a test of bleeding of the gums, (2) saliva hemoglobin—an indicator of inflammation, and (3) monocytogram which is a standard test for blood in the saliva and involves checking levels of three different types of cells: promonocytes, monocytes and polymorphonuclear cells. These tests were repeated twice, once before treatment with the ultra-disperse particles, but after a standard course of treatment (indicated as before treatment in the second column of the table in FIG. 15), and once after treatment with the ultra-disperse particles (indicated as after treatment in the second column of the table in FIG. 15). In all tests an improvement was seen, in the capillary resistance test the gums were able to withstand a pressure over a longer period of time, and in the other two tests lower levels of bleeding were recorded.

Often a desired treatment is accompanied by a negative side effect. A particle can be used to bind the chemical in such a way that it can interact with the other surface but remains attached to the particle and can be flushed away. This allows a chemical to be present with partial chemical participation or even without direct chemical participation. For example, iodine is an effective bactericide with a drying side effect. By binding the iodine to a particle the bactericidal properties can be isolated from the drying properties.

In another preferred embodiment the particle is provided as an at least dual action particle which causes a reaction and then deals with the results of that reaction (mechanism Y6). Since it is known that the biological system will respond aggressively, a component is included to neutralize and absorb the response of the system. The particle is responsible both for activation and deactivation of the system's toxic response. For example, the particle can be used as a carrier to reduce the side effects of antibiotics. The particle is directed to the microbes so that very low doses of antibiotic are necessary as it is localized at the source of the problem. In this case the low dose antibiotics effect a high local concentration. Because of the directed action conventional medicines can be used at the concentration levels of alternative medicines. The dual action is the absorption of the toxins released as a result of the action of the antibiotics. The particle can be used to carry any of a number of different types of additives including antibiotics and other medicines (including anti-cancer agents), vitamins, microelements and to effect their proper distribution in the biological medium.

The particle can be provided as a hydrophilic powder mixed in an oil base, providing a completely water-free environment. A bacterium which enters this oil will be instantly dehydrated without being able to release its toxin. A hydrophobic powder in a water base will also kill the bacterium by pulling the oil out of it, thereby destroying the cell membrane. However, this will release the toxic contents of the cell into the surrounding environment.

In another example, a particle is used in UV water sterilization. In current methods of UV sterilization a UV light is directed through water in order to kill any microbes found in the water. Water is normally transparent to UV light but the presence of microbes blocks the light so that the UV cannot penetrate past the first layer of microbes. Use of a particle with properties to scatter the UV light allows the UV to penetrate more deeply into the water and more effectively sterilize the water. The dual action of the particle is its ability to absorb the result of the sterilization, the dead microbes.

In yet another example, a particle is used for radiation absorption. With radiation exposure there is a need to protect the cell. For this type of application, a sunblock cream has been created with a dual action. When UV light from sunlight is absorbed by the skin free radicals are produced. The sunblock cream which absorbs the UV radiation energy can be provided with a particle which releases an electron by photoeffect to transform the free radical. The energy which would have been used to damage the skin and cause it to age has been transformed to promote skin renewal. In this way the particle has been prepared for the expected results.

This can be used in all types of radiation. For use in cancer treatment a particle is engineered to selectively reach the cancer cells and once there to absorb radiation in high amounts creating a high temperature to burn off the cancer cells. The dual action provided allows the particle to absorb the toxins released by the death of the cells. By using these particles the radiation is focused and therefore higher levels of radiation can be used safely with less injury to the patient.

In a toothpaste application a hydrophilic particle is provided which breaks the adhesive connection between the plaque and the enamel of the tooth in a non-abrasive fashion without the need for fluoride which is the current active ingredient of most toothpaste and is known to be toxic. Plaque colonies tend to aggregate by the salivary glands where phosphates are released. Calcium phosphate acts as a bridge between plaque colonies and the enamel on the tooth. A toothpaste is provided which is water-based with hydrophilic particles mixed in and with cells of dry hydrophobic particles. When the toothpaste is used, the hydrophilic particles activate the water so that it is able to dissolve the phosphate and release the plaque. The hydrophobic particles will absorb both the plaque that is being released and the toxins released by the death of the bacterial colonies. The addition of a negatively charged particle allows simultaneous treatment of inflammation caused by gum disease, as seen in FIG. 15. Because of the hydrophobic properties, this toothpaste will not coat the inside of the mouth as current toothpastes do. The particles have a non-abrasive polishing effect. Fluoride need not be used or can be used in very low concentration attached to a hydrophobic particle for direct delivery to the enamel of the tooth. The enamel's high affinity for fluoride will cause the release of the fluoride only in the vicinity of the enamel.

For a completely non-abrasive dentrifice, the particles in the toothpaste described above would be provided in a chewing gum with a swelling component to absorb the released plaque. Because of the small size of the particles, they can reach places a normal toothbrush cannot. Since they work on the chemical bond between the plaque and the enamel, there is no need for a toothbrush to provide abrasion. In addition, the gum is single use and therefore provides a clean method of cleaning the teeth, unlike the toothbrush which is a surface for microorganisms to grow on between uses. Using the gum, one can brush their teeth at any time. It can save time in the morning, as one can use the gum during the commute to work.

The particles can be used in a liquid base as a hygienic body wash in all body cavities, including surgical cavities.

There are many cosmetic applications of the dual action embodiment. Among them, an exfoliant cream is provided which both peels and absorbs the dead skin. A cream for melting skin oil for extraction of oil from skin pores without damage is provided. A chemical is used to lower the melting point of the oil allowing it to flow out of the skin pore, and in combination with this chemical a hydrophobic component is provided for absorbing the oil providing effective cleaning.

Particles can be used in many other applications, such as agriculture. A particle is provided which coats UV-sensitive bacteria to protect them and allow them to be used as biological exterminants.

In another embodiment, the particle can be provided with a multilevel slow-release mechanism as a particle which has a number of layers of active ingredients encapsulated in slow-release coatings. In this fashion, a multi-level action can be programmed with active ingredients being released in sequence and the final active ingredient being programmed to absorb the results of the reaction.

Research has shown particles modified to have specific chemicals on the surface are effective in treatment of specific disorders, as shown in FIG. 16. For example. CaF₂ is effective in the treatment of scars and keloids. Calcium and fluoride act selectively on the connective tissues which make up the scar tissue making them less dense and eventually dissolving them.

Pruritis Senilis is a condition in which at ages above 60, magnesium becomes less prevalent in the skin, causing skin dryness and itching which is not accompanied by a rash. This condition can be alleviated by using particles to add back the missing magnesium.

In a condition known as Cuprosis, microdoses of particles modified to contain BaCO₃ improve the mineral exchange and act on the endocrine system, lowering the hypertonic pressure in the walls of the blood vessels, improving blood circulation.

Acne Vulgaris is a common problem especially in the teenage years when hormonal imbalances occur. Acne is accompanied by scarring of the tissues surrounding the follicles. In the follicles and oil glands, blood vessels expand and lymph fluid accumulates. The surrounding tissue absorbs plasma causing swelling and blocking the follicles from releasing their contents, allowing microorganisms to grow and pussy secretions to be trapped in the follicle. Use of sulfur and SiO₂ accelerates the opening of the follicle allowing release of its contents. In addition, use of CaS has the effect of sulfur with the added effect of calcium to dissolve scar tissue (see above).

Use of particles modified to contain AgNO₃ on small scratches and fissures has a local disinfectant effect and aids in blood clotting while having a cauterizing effect on the tissues. Particles modified with AgNO₃ structurize the secretions from the wound such that microorganisms cannot penetrate and allowing for quicker healing. This is helpful in diabetic patients in whom the healing process is especially slow.

Patients who suffer from balding caused by alopecia can be helped with a particle modified to deliver zinc. Heavy metals such as zinc are known to improve the functioning of the nervous system. Lack of zinc in an organism can be seen in a lack of hair follicle growth and functional impairment of nerve endings. This also causes the hair to be more fragile and breakable and to grow more slowly. With the use of a particle modified to deliver zinc, the problem of alopecia can be treated.

In summary, the present invention provides an infinite number of types of modified ultra-disperse particles for use in an unlimited number of applications in many fields including, but not limited to, pharmaceuticals, cosmeceuticals, agriculture and food industry.

Having described the invention with regard to specific embodiments thereof, it is to be understood that the description is not meant as a limitation, since further modifications may now suggest themselves to those skilled in the art.

1. A biological media structure comprising:
an ultra-disperse nano-particle of a hydrated oxide;
a biological tissue; and
surrounding media,
said structured biological media comprising a three-sided biological system.

2. The structure of claim 1 wherein said particle has a substantially spherical shape.

3. The structure of claim 1 wherein said particle has selective electrical attraction to areas of charge anomaly on said biological tissue surface, so as to coat said areas by providing said stable three-sided biological systems preventing toxin penetration through said tissue surface.

4. The structure of claim 1 wherein said particle has selective electrical attraction to bacterial surfaces, so as to coat said surfaces by providing stable three-sided biological systems preventing bacterial activity including ion or other exchange through the membrane.

5. The structure of claim 1 wherein said particle is provided in a powdered form.

6. The structure of claim 5 wherein said particle is provided pressed into a pill with an anti-aggregation component for dispersal of said powder upon digestion.

7. The structure of claim 1 wherein said particle is provided in a porous bag for submersion in water, said porous bag preventing escape of said particles when dry so as to prevent inhalation of said particles.

8. The structure of claim 1 wherein said particle is provided in a capsule with dissolvable walls for use in at least one of swallowing and dissolving in water.

9. The structure of claim 1 wherein said particle has a modified surface structure.

10. The structure of claim 9 wherein said particle surface has hydroxyl groups having undergone partial methylation, providing a particle with a surface having methylated sites, said particle surface being partially hydrophobic and partially hydrophilic.

11. The structure of claim 10 wherein said particle produces an IR spectrum with a peak at 3750 nm showing percentage hydrophilicity and a peak at 2980 nm showing percentage hydrophobicity.

12. The structure of claim 1 wherein said particle is provided in a mechanical mixture of hydrophobic and hydrophilic particles, producing an IR spectrum with a peak at 3750 nm showing percentage of hydrophilic particles and a peak at 2980 nm showing percentage of hydrophobic particles.

13. The structure of claim 10 wherein said particle is provided with approximately 10%-90% hydrophobic surface groups, resulting, conversely in approximately 90%-10% hydrophilic surface groups.

14. The structure of claim 10 wherein said particle is provided with an electrical potential affecting ion channels in said biological tissue.

15. The structure of claim 14 wherein a plurality of said particles form a helical structure.

16. The structure of claim 10 wherein said particle provides a stable thixotropic water-oil emulsion.

17. The emulsion of claim 16 further aerated to provide a water-oil-gas emulsion.

18. The structure of claim 10 wherein said particle is etched with interconnected interior channels etched into it causing extremely high surface area per unit solid mass.

19. The structure of claim 18 wherein said particle surface has a ratio of hydrophilic to hydrophobic surface groups between approximately 0.1 to 0.3.

20. The structure of claim 18 wherein said particle interconnected interior channels are filled with a component for slow release into said three-sided system.

21. The structure of claim 18 wherein said particle interconnected interior channels are further etched causing said particle to disintegrate into even smaller nano-particles of under approximately 10 nm in size.

22. The structure of claim 21 wherein said particle in said smaller nano-particles are provided with a hydrophilic to hydrophobic surface group ratio of between approximately 0.4 to 0.8.

23. The structure of claim 10 wherein said remaining hydroxyl groups on said particle surface are further modified so as to control at least one of surface charge, pH and electrical potential.

24. The structure of claim 23 wherein said further modifications are provided as protrusions from said particle surface.

25. The structure of claim 24 wherein said particle protrusions are composed of the same chemical composition as said particle.

26. The structure of claim 24 wherein said particle protrusions are composed of a different chemical composition than said particle.

27. The structure of claim 24 wherein said particle protrusions are composed of at least one of metals, nonmetals, macromolecules, antibiotics, vitamins, microelements, and organic material.

28. The structure of claim 24 wherein said particle protrusions are branched in shape.

29. The structure of claim 28 wherein said particle protrusions have multiple branching sites.

30. The structure of claim 28 wherein said branched particle protrusions are composed of the same chemical composition as said particle.

31. The structure of claim 28 wherein said branched particle protrusions are composed of a different chemical composition than said particle.

32. The structure of claim 31 wherein said particle protrusions are composed of multiple chemical compositions, each composition layered sequentially on a protrusion formed previously.

33. The structure of claim 32 wherein said particle provides a three-dimensional electrical charge spatial template in said media, as determined by said multiple chemical compositions.

34. The structure of claim 24 wherein said particle protrusions are attached to said particle by a low bonding force such that said protrusions can be detached upon treatment by at least one of exposure to high intensity ultrasound waves and insertion into liquid.

35. The structure of claim 34 wherein said detachable particle protrusions create nano-particles of under approximately 10 nm in size.

36. The structure of claim 34 wherein said detachable particle protrusions form an electrostatic interaction.

37. The structure of claim 24 wherein said methylated sites of said particle are demethylated and a second set of protrusions of an opposite charge from the first set of protrusions is added, so as to form a particle with two sets of protrusions of opposite charges.

38. The structure of claim 1 wherein said particle has acquired a charge through a double electric layer so as to be capable of electrostatic interaction with regions of a third component.

39. The structure of claim 1 wherein said particle is capable of charge reversal according to the pH of the environment.

40. The structure of claim 1 wherein said particle is used for directed action on microorganisms of different types.

41. The structure of claim 1 wherein said particle is capable of interaction with at least one of affected cell regions or bacteria, while said particle retains high absorption capacity and selectivity.

42. The structure of claim 1 wherein said particle is capable of adsorbing the toxic substances formed as a result of vital activity and decomposition of a biosystem.

43. The structure of claim 1 wherein said particle is provided with dual action, such that any biological function caused by the presence of said particle is followed by a process of at least one of toxic result neutralization, absorption and destruction.

44. The structure of claim 1 wherein said particle is characterized by a broad interaction spectrum, from intermolecular to chemical, with at least one of the environment and the boundary of any system located in it.

45. The structure of claim 1 wherein said particle exhibits, on appearance of a third component of said three-sided biological system, active, self-organizing properties, thereby responding adequately and selectively to the appearance of said third component and to its charge state, thereby forming a localized stable three component system.

46. The structure of claim 1 wherein said particle exhibits selectivity of particle action depending on the size and shape of an object, on the charge, on the hydrophilic-hydrophobic pattern and on the availability of functional groups.

47. The structure of claim 1 wherein said particle enables structurization of the bioenviromnent with formation of at least one of locally non-homogeneous regions and nano-size fluctuations, interacting through a network of three dimensional bonds containing an inorganic particle.

48. The structure of claim 1 wherein said particle forms said three-sided biological system in which said surrounding media comprises structured thixotropic biofluids, said system acting as an analog of membranes impeding the transport of bacteria, of their nutrients and of dissolved inorganic compounds and ions.

49. The structure of claim 48 wherein said particle forms a stable three-dimensional structure in a thixotropic environment when in contact with an inanimate component and forms an unstable structure which has variable thixotropy when in contact with a living component.

50. The structure of claim 1 wherein said particle is provided with the capacity to be adsorptive and chemisorptive and to form chelates allowing inorganic and organic components to be isolated.

51. The structure of claim 50 wherein said particle has adsorptive capacity for interaction with hydrophobic-hydrophilic regions of bio-objects as well as for specific interaction with components of a living environment.

52. The structure of claim 1 wherein said particle is a combination of positively and negatively charged particles provided for encapsulation of bacteria.

53. The structure of claim 10 wherein said particle has said hydrophilic particles that are used to inactivate bacteria inside a block of structurized water, with practical disruption of the link between the bacteria and the environment.

54. The structure of claim 10 wherein said particle is used for and at least one of intermolecular interaction with hydrophobic regions of membranes, supply and removal of oils.

55. The structure of claim 10 wherein said particle is provided with a specific hydrophobic-hydrophilic balance on the surface permitting formation of a branched three-dimensional network in a system of non-interactive hydrophobic-hydrophilic environments across the surface of a solid body.

56. The structure of claim 10 wherein said particle is provided with a given hydrophobic-hydrophilic balance on the surface causing chemical reactions over specific surface hydroxyl groups with metal chlorides, creating highly non-uniform heterogeneous environments with new thixotropic properties, different charges, different photochemical abilities and other changed properties.

57. The structure of claim 24 wherein said particle is formed with a series of layers of active ingredients which are encapsulated in slow-release covers.

58. The structure of claim 52 wherein said particle has active ingredients that are released in sequence and a final active ingredient absorbs the results of the reaction.

59. The structure of claim 24 wherein said particle protrusions form a “lock and key” system whereby an ionic channel is shut, encapsulating a microbe and shielding it from the environment.

60. The structure of claim 10 wherein said surface hydroxyl groups are replaced with at least one of inorganic radicals, and organic radicals including the group of amines, alcohols, iodine, and bromine, leading to formation of bonds of the donor acceptor type, complexes with coordination type charge transfer, covalent bonds and dispersion interaction with the functional radicals of a bio-object.

61. The structure of claim 24 wherein said particle is provided in mechanical mixtures of said particles which are differently charged in the presence of water, depending on the pH of the environment, and therefore will interact differently with each other and with specific biomembrane regions.

62. The structure of claim 24 wherein said particle is subjected to mechanical mixing, followed by settling of substances with heterogeneous structures in an aqueous environment leading to formation of xerogels with an ultra-heterogeneous pore structure.

63. A method of modifying the surface of ultra-disperse nano-particles of hydrated oxides by partial methylation, said method comprising the steps of:
heat-treating said particles in an open vessel at an appropriate temperature so as to remove absorbed and bound structural water;
reacting said heat-treated particles with functional organic molecules in the gaseous phase at high temperature so as to methylate surface hydroxyl groups;
removing excess reagent and reaction products;
hydrolyzing unreacted chloride groups on said surface through heating in the presence of saturated water vapor;
heating finally in at least one of an open vessel and an inert atmosphere; and
cooling,
such that said nano-particles become modified by partial methylation of their surface.

64. The method of claim 63 wherein the step of reacting said heat-treated particles is allowed to continue for between approximately 5 and 60 min at a temperature of approximately between 200-300° C., the length of said exposure determining percentage of said methylation.

65. The method of claim 63 wherein the step of hydrolyzing is effected at a temperature of between approximately 250-300° C. for a period of approximately one hour.

66. The method of claim 63 wherein said final heating step is effected at a temperature of approximately between 200-300° C.

67. The method of claim 63 further comprising the step of checking percent methylation by IR spectroscopy, said hydroxyl groups appearing at approximately 3750 nm and said methyl groups appearing at approximately 2980 nm.

68. The method of claim 63 further comprising the step of:
etching non-methylated surface sites so as to create interconnected interior channels providing said particles with high surface per unit mass.

69. The method of claim 63 further comprising the step of further modifying said partially methylated particles by building spike-like protrusions on said surface of said particles in areas which have not been methylated, by heat-treating in the presence of a desired component.

70. The method of claim 69 further comprising the step of monitoring control of growth of said protrusions by measuring the intensity of said hydroxyl group peak at 3750 nm through IR spectroscopy.

71. The method of claim 69 wherein said spike-like protrusions are comprised of at least one of SiO₂, Al₂O₃ and TiO₂.

72. The method of claim 69 wherein the step of heat-treating in the presence of SiO₂ takes place at a temperature of at least one of 200° C., 400° C. and 650° C.

73. The method of claim 69 wherein the step of heat-treating in the presence of at least one of Al₂O₃ and TiO₂ takes place at a temperature of between approximately 200-400° C.

74. The method of claim 69 further comprising the steps of:
heating said particles to between approximately 500-700° C. so as to demethylate said methylated areas of said particle surface, thereby also methylating said spike-like protrusions so as to form a protective cap; and
building a second type of protrusion on said demethylated areas, by heat treatment in the presence of a second component,
so that a second type of protrusion is formed on said demethylated areas.

75. The method of claim 74 further comprising the step of:
reiterating partial methylation of said particle so as to produce branched protrusions.

76. The method of claim 63 further comprising the steps of:
creating drops of approximately 50-100 microns with an ultrasound atomizer,
feeding said drops into a chamber with a layer of said hydrophobic particles such that said drops become coated by said particles due to collision forces; and
introducing said coated drops into an emulsion under turbulent mixing,
such that said hydrophobic particles will allow insertion into an oily medium, resulting in an emulsion with a high water content.

77. The method of claim 76 wherein said step of introducing said coated drops takes place in a gas enriched environment so as to create a water-oil-gas emulsion.

78. The method of claim 77 wherein said gas is at least one of air and ozone.

79. The structure of claim 10 wherein a combination of partially hydrophilic and hydrophobic particles are provided in a toothpaste.

80. The structure of claim 79 wherein said partially hydrophilic particles break the adhesive connection between the plaque and the enamel of the tooth.

81. The structure of claim 80 wherein said partially hydrophobic particles adsorb the plaque released by said partially hydrophilic particles.

82. The toothpaste of claim 79 further comprising particles with a negative electrical charge for treatment of inflamed gum tissue.

83. The hydrophobic particle of claim 79 further comprising flouride for direct delivery to the tooth enamel.

84. The toothpaste of claim 79 further comprising flouride.

85. The toothpaste of claim 79 wherein said partially hydrophilic and said partially hydrophobic particles comprise less than approximately 20% of the total weight of said toothpaste.

86. The structure of claim 10 wherein said particle is provided as a combination of hydrophilic and hydrophobic particles that are provided in a chewing gum for use as a dentrifice.

87. The structure of claim 10 wherein said particle is for use in medicinal applications.

88. The structure of claim 10 wherein said particle is for use in cosmetic applications.

89. The structure of claim 10 wherein said particle is for use in hygiene applications.

90. The structure of claim 10 wherein said particle is for use in the food industry.

91. The structure of claim 10 wherein said particle is for use in agricultural applications.

92. The structure of claim 10 wherein said particle is for use in water treatment applications.

93. The structure of claim 10 wherein said particle is for use in disinfection applications.
